(19) United States
(12) Patent Application Publication
(10) Pub. No.: US 2008/0045809 A1
(43) Pub. Date: Feb. 21, 2008

(54) Devices for measurement of medical and physiological parameters using mass storage device interfaces

(75) Inventor: Kormakur Hlini Hermannsson, Herning (DK)

Correspondence Address: BIRCH STEWART KOLASCH & BIRCH, PO BOX 747, FALLS CHURCH, VA, 22040-0747, US

(73) Assignee: Nox Medical, Reykjavik (IS)

(21) Appl. No.: 11/889,849

(22) Filed: Aug. 16, 2007

Related U.S. Application Data
(60) Provisional application No. 60/837,941, filed on Aug. 16, 2006.

(51) Int. Cl. A61B 5/00

(52) U.S. Cl. 600/300

(57) ABSTRACT

Medical and physiological measurement systems for diagnosis analysis are provided, in particular for sleep and blood pressure diagnosis, which are given by simple and cost efficient designs, wherein the corresponding medical devices and/or medical data interface devices are equipped with such data communication ports that they appear as mass storage devices upon connectivity with a computer. The data transparency offered by such standardized data transfer means is implemented to offer, for example, the possibility of storing diagnosis software in a platform independent manner in memory of the mass storage device. Furthermore, analysis results, diagnosis related reports and/or raw data collected by the medical system can easily be accessed, viewed and/or analyzed by the measurement system operator at the computer by accessing the corresponding devices as mass storage devices.

### AREA OF INVENTION

The present invention relates to medical and physiological measurement systems allowing for diagnosis analysis, in particular for sleep and blood pressure diagnosis, by simple ways of operation and at low cost enabled by mass storage device connectivity.

### BACKGROUND OF THE INVENTION

Optimization in healthcare requires technology to be increasingly used in patient diagnostics. By doing simple medical and physiological measurements it becomes possible to make referrals which are based on well substantiated information and thereby lead to well focused and more efficient diagnosis results. This is especially important in long term monitoring, such as sleep diagnostics, where the patient is unaware of his condition and therefore unable to provide accurate information. It may consequently often be possible to bypass the necessity of involving the patient in otherwise required but expensive and complicated treatments or studies.

Despite these obvious benefits, the efficiency of currently available medical and physiological measurement systems, hereinafter referred to as medical systems, are known to be limited by two attributes, namely by either being very complicated and expensive or oversimplified and inaccurate. The more complicated medical systems typically require a computer software application for synchronizing, reviewing and analyzing acquired data gathered by measurement devices and for generating corresponding reports. Such software applications are known to be highly complex and associated with expensive technical support needed for setup, maintenance and personnel-training. The continuous advancements in the computer environment require the medical software to be capable of running in inconsistent environments, using different types of operating systems, different constellations of accessories drivers, different versions, etc. It follows that the medical software must be continuously maintained and verified to run correctly in the different environments. Due to safety requirements in medical development, this maintenance is required even after release of the software and it often results in software updates at the customer site, causing customer inconvenience, support cost and periods of down-time when the medical device is non-functional due to maintenance. It follows that the development and service cost of medical systems using conventional computer applications can be quite substantial, in particular when features provided by the underlying operating system, such as printing capabilities or display features, are being used. Consequently, the price of such medical systems is driven high enough to cover all costs, often in low volume markets, which eventually may defer potential customers away from buying.

The simpler and lower cost solutions avoid these complications by completing processing and visualization of results onboard the measurement device. Due to size limitations, power requirements and other factors, the approach is most often based on displaying a single parameter on a display with very limited review possibilities. This has been criticized by the medical community as imposing a “black-box” approach, giving the medical practitioners no opportunity to review prerequisites leading to the diagnosis conclusion. The limited onboard processors are most often incapable of computing and delivering accurate results, also since working with large datasets can be very problematic in embedded systems. The consequences are limited accuracy, imprecise analysis, limited delivery of parameters and limited possibilities of comparing parameters originating from different measurements due to lack of concise electronic reporting. It follows that overall diagnosis conclusions may represent unreliable diagnosis results.

The following publications give an overview and discussion on topics related to this field:
1. Ferber, R A, Millman, R P, Coppola, M P, et al ASDA standards of practice: portable recording in the assessment of obstructive sleep apnea. Sleep 1994; 17,378-392[ISI][Medline],
2. American Sleep Disorders Association. Practice parameters for the indications for polysomnography and related procedures: Polysomnography Task Force, American Sleep Disorders Association Standards of Practice Committee. Sleep 1997; 20,406-422[ISI][Medline],
3. Chesson, A L, Ferber, R A, Fry, J M, et al The indications for polysomnography and related procedures. Sleep 1997;20,423-487[ISI][Medline],
4. Ross S D, Allen I E, Harrison K J, et al. Systematic review of the literature regarding the diagnosis of sleep apnea: evidence report/technology assessment No. 1. Rockville, Md.: Agency for Health Care Policy and Research; February 1999; AHCPR Publication No. 99-002.

### SUMMARY OF THE INVENTION

The object of the present invention is to provide improved medical systems for avoiding the complications, as described above, found to be inherent in current medical systems. For this purpose, the medical system defined according to this invention is capable of collecting measurements taken from a patient or at a patient's location, where measurements taken from a patient are provided by sensors attached to the patient's body and measurements taken at a patient's location are given by sensors located in the patient's proximate environment, and processing the full acquired measurement dataset, for making accurate diagnostics, and it offers possibilities for generating and providing electronic analysis and reports containing extensive sets of appropriate parameters, graphs and/or other visualizations of diagnostic results and/or data. Furthermore, for lowering costs and complexity, the medical system according to this invention is designed with the aim of operational independency in regard to the continuously changing computer environment.

In one of the embodiments of a medical system defined according to the present invention, the medical system comprises at least one medical device defined according to the invention, which collects measurement data taken from a patient or at a patient's location, and stores these in at least one memory component comprised by the corresponding medical device for instant and/or later data processing and/or analysis. The respective processing of data for diagnosis purposes can take place in the medical device itself, by use of an inbuilt microprocessor, or in a software application which runs on a computer. The medical device according to the invention allows connectivity with a computer via a data communication port, whereas when connected to the computer, the medical device appears as an MSD (Mass Storage Device) on the computer, allowing direct access to collected measurement data and/or data processing results stored and/or generated by the medical device.

By using the MSD interface the communication taking place between the medical device according to this invention and a computer is supported by standard and consistent protocols of data transfer, based on well known features provided by most commercially available computer platforms, and can therefore be considered as being largely independent on the underlying computer environment. The MSD interface approach allows all kinds of data, files and computer software to be accessed in the device the same way as if it was stored on the computer hard-disk or CD-ROM. By this approach, the invention allows the user to open or generate reports on standard formats as soon as the device has been connected to the computer, making it possible to avoid the usual steps of running pre-installed computer software that uploads recorded data for visualization and depends on independent execution of scoring procedures.

The medical device according to this invention allows accessing data and/or data processing results stored in the memory of the medical device, by having the medical device connected to a computer in such a way that it appears as an MSD on the computer. Consequently, the medical device according to this invention allows the implementation of medical systems which are simpler to maintain and use, and are therefore subject to lower costs than other medical systems which have been available up to now. In contrast to other approaches of simplification, the medical system based on this invention is not simplified at the cost of reduced quality in diagnosis or representation.

The above mentioned microprocessor comprised by the medical device of this invention can be selected to be powerful enough to perform data processing on data stored in the medial device's memory, for generating analyses and/or reports which are also stored in the medical device's memory. Such analysis and/or reports can simply be accessed by the respective computer which is connected to the medical device and views its content as an MSD. By this approach the medical system can easily be designed to achieve computer platform and software independency, lowering the corresponding complexity of implementation, costs of use and maintenance. Furthermore, display and/or input elements can be implemented in the medical device of this invention, for allowing displaying data, analysis results and reports without the use of other data processing units such as computers.

It is furthermore possible to store computer software applications in the memory of the medical device given according to this invention, which can be accessed and executed by the respective computer which is connected to the medical device and views its content as being an MSD. Such computer software applications can be used, for example, for data analyzing, report generation, viewing, synchronizing or manipulating data stored in medical devices, such as measurement data, results, reports or various parameters used for configuring the medical device or settings of diagnosis procedures.

Another object of this invention offers similar functionalities but represents a modification of the medical system which uses medical devices according to the present invention, wherein the medical devices are replaced by a combination of components, comprising at least one medical data interface device which can be connected to the respective computer and appears on the computer as an MSD. The medical data interface device furthermore comprises an interface to one or more smart sensors. The smart sensors comprise a combination of a sensor and additional components required to perform and store measurements in a memory component comprised by the smart sensor. The smart sensors can furthermore comprise a microprocessor for pre-processing measurements and they offer data transfer via a comprised communication port. By these means, the smart sensors can be organized in a distributed manner on a patient or at a patient's location for collecting, storing and possibly pre-processing measurement data. By this way, the medical data interface device collects and stores measurement data and/or pre-processed measurement data from the smart sensors in its comprised memory component for instant or later data processing and/or analysis, for example by a microprocessor comprised by the medical data interface device. The medical data interface device can comprise a display and/or input elements for allowing display of data, analysis results and/or reports without the use of other data processing units such as computers. The medical data interface device can store computer software applications in the medical data interface device's memory which can be accessed and executed by the respective computer which is connected to the medical data interface device and views its content as being an MSD. Such computer software applications can be used, for example, for viewing, synchronizing and/or manipulating data stored in the medical data interface device, such as measurement data, results, reports or various parameters for configuring the pre-diagnosis system or settings of pre-diagnosis procedures.

Other possible embodiments according to the present invention are introduced in the description of preferred embodiments.

### BRIEF DESCRIPTION OF DRAWINGS

FIG. 1 shows an embodiment of a medical system based on the use of medical devices of this invention.

FIG. 2 shows an embodiment of a medical system based on the use of a medical data interface device and smart sensors according to this invention.

FIG. 3 shows an embodiment of a medical system based on the use of a medical device and smart sensors according to this invention, which are continuously connected by a wireless link.

FIG. 4 shows an embodiment of a medical system based on the use of an extended medical device and smart sensors according to this invention.

FIG. 5 shows an embodiment of a medical system based on the use of an extended medical device and smart sensors according to this invention, which are continuously connected by a wireless link.

### DESCRIPTION OF PREFERRED EMBODIMENTS

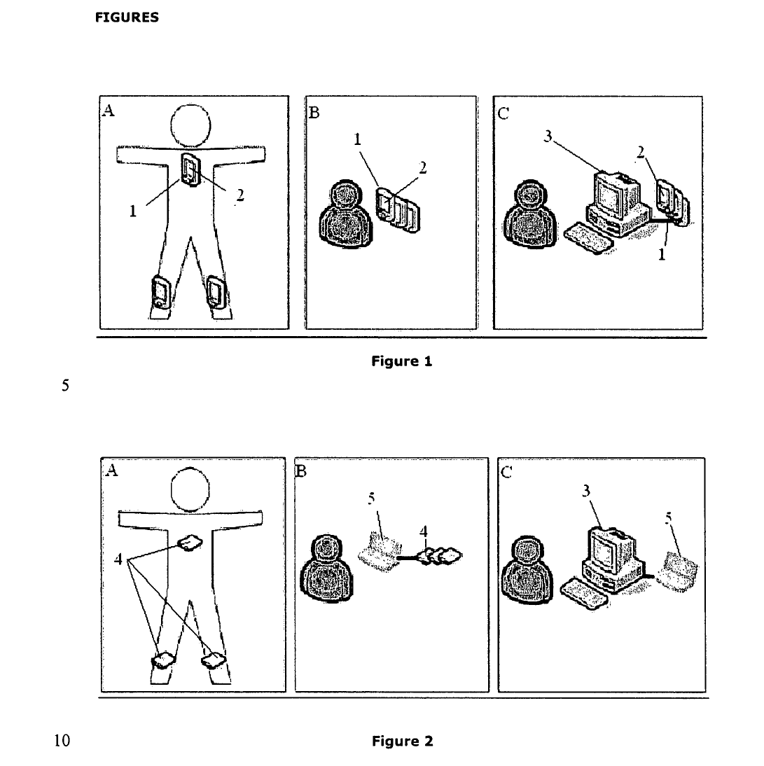

Referring to the embodiment illustrated in Figure la, the first embodiment of a medical system according to this invention is to have at least one medical device (1) of this invention, which each contains or is connected to at least one sensor, such as for example a respiratory effort belt sensor, body position signal/activity sensor, light detection sensor, snoring sound sensor, snoring vibration sensor, pressure cannula sensor, mask pressure sensor, EMG sensor, LM Vibration sensor, ECG sensor, pulse oximeter, EEG/EOG sensor, flow generator digital or analog output sensor, bruxism sound/vibration sensor, blood pressure sensor or any other sensor recognized by a person skilled in the art to be applicable for measuring signals taken from a patient or at a patient's location for diagnosis purposes. Such signals can include for example ECG (electrocardiogram), eeg (electroencephalogram), SpO2 (oxygen saturation), respiratory effort and flow signals, body activity signals, body position signals, blood pressure signals and could be applied for analyzing syndromes such as apnea/hypapnea, respiratory flow limitation, RLM, snoring, activity, heart rate variations, high/low blood pressure for diagnosis purposes, such as for example for detecting and/or evaluating quality of sleep and/or sleep disorders. Such measurements are recorded and stored, at least partly, by each medical device (1), possibly in a pre-processed format, in a memory component, such as for example DRAM. SRAM, NVRAM, memory cards inserted in a respective memory card slot, or other possible memory types known to a person skilled in the art, which is comprised by each medical device (1). The aforementioned pre-processing of measurement data can, for example, involve signal filtering, noise cancellation, event counting, compression or any other related pre-processing task of measurement data known to a person skilled in the art. Each medical device (1) comprises at least one microprocessor for supporting various tasks such as sampling of above mentioned data, the aforementioned pre-processing of measurement data, data processing, data storing, control of data transfer and/or mass storage data device computer interfacing, visualization etc. This microprocessor can be selected powerful enough to perform complex analysis of respective collected data and to generate results, such as analysis and/or reports which are stored in electrical form in the respective memory of each medical device (1). Such analysis and/or reports could contain data related to various medical and physiological measurements for various diagnosis reasons such as for example count of apnea events, count of oxygen desideration, trend view of blood pressure, sleep reports, ECG reports, epilepsy, etc. As shown in FIG. 1, the medical device (1) can also comprise a display (2) for visualization, such as for example an LCD or TFT-LCD display; which can be used by the operator to review data, analysis and results, and possible reports which have been generated and/or stored in a medical device's (1) memory.

As illustrated in FIG. 1b, the data which is relevant for the diagnosis purpose can be found, in the case of a plurality of medical devices (1), distributed in the memory of many medical devices (1). In case of displays (2) being comprised by the medical devices, the operator can view, on the display (2) of each medical device (1), data, analysis and results, such as possible reports, collected and/or generated in the corresponding medical device (1) and stored in the memory of the same medical device (1). For the purpose of selecting data, analysis and/or results to be viewed on the respective display (2), the medical devices (1) can also comprise input elements for receiving inputs from the operator and therewith enabling interactive and/or selective viewing. Such input elements, as referred to in this invention, can be based on any conventional technology, such as buttons, keys, touch-pads, touch-screens or voice recognition means, to name a few possibilities.

The medical device (1) furthermore comprises a data interface which is used for connecting the medical device (1) to a computer (3). In this sense the computer can be comprised by data processing unit, such as a Personal Computer, Workstation or any other type of digital computing devices which may be suitably selected to accomplish the underlying computing tasks. When connected to a computer (3), the medical device (1) acts as a USB memory stick or USB, FireWire or Ethernet connected hard disk, external CD drive or any other possible type of a Mass Storage Device (MSD) accessible by the computer (3). This requirement on the medical device (1) is met by selecting it's comprised microprocessor to be powerful enough to run the necessary services for enabling the MSD connectivity, that is to handle both the onboard data processing and simultaneously the mass storage device interface towards the computer, including the file system access to the data stored in the medical device's (1) memory.

After collecting and processing measurements in the medical devices (1), each medical device (1) can be accessed by the computer (3) as illustrated in FIG. 1c and viewed as an MSD, and the operator finds each to contain diagnosis study results in form of electronic data, analysis and/or reports. The electronic data, analysis and/or reports can be stored using standard computer data formats such as TXT, RTF, HTML, PDF or XML, and is therewith ready to be viewed, copied to the computer, printed or opened for editing by use of commercial computer software applications. Depending on the selected type or types of electronic data format, the medical device's (1) memory must be dimensioned accordingly, such that sufficient memory is available for storing the desired amount of electronic data, analysis and/or reports using the corresponding at least one selected data format.

To allow for more sophisticated data analyses to be performed than can be implemented within the medical device (1), the computer (3) can feed the electronic data, analysis and/or reports provided by the medical devices (1) according to FIG. 1c, into a computer software application for further data processing. This allows the full power of the computer (3) to be used for analyses and report generation, without compromising the simplicity of the operating procedure. Such data processing can include the execution of complicated and sophisticated diagnosis algorithms, combining by synchronization and extraction by evaluation, information which is inherent in results stored by the medical devices (1) in form of electronic data, analysis and/or reports. The respective computer software application can be stored either in the computer (3) or in a medical device's (1) memory, whichever may be more appropriate for each case of a medical application. For the case that the computer software has been stored in the medical device's memory (1), it can be configured such that upon connecting the medical device (1) to the computer (3) as an MSD, according to FIG. 1c, the respective computer software application is automatically executed by the computer (3). The steps of executing the software, opening data folders, performing analyses and generating electronic data, analysis and/or reports can easily be automated and hidden from the user to maximize simplicity without impairing the quality of analysis.

By this constellation the overall medical system and procedure represents a very effective and efficient means to collect relevant information from a patient or at a patient's location and to process these for gaining diagnosis results in the medical devices (1) and/or in software applications which runs on the connectable computer (3). Consequently, the respective computer software application can be designed in an efficient manner, offering transparent functionalities which are easy and simple to learn, manage and use by the corresponding operator. These software application features may include, for example, processing of large datasets, synchronizing data and/or medical devices, viewing data on computer monitors or storing data in appropriate memory locations.

By this approach the above described system does solve many of the problems inherent in conventional medical diagnosis systems of today. The medical system according to this invention does not necessarily require computer software application installation and training, but is still capable of providing the user with a sophisticated diagnostic result and electronic reporting. This automation greatly simplifies the operating procedure for the user, allowing new users to perform professional and sophisticated diagnostics with minor efforts invested in system operation related specialization and training. The main benefits are lower installation costs, lower training costs and overall easier operation. It follows, that the medical system according to this invention represents an efficient and effective system which can easily and in a cost efficient manner be implemented, maintained and understood by respective technicians and/or operators.

FIG. 2 illustrates a second possible embodiment of the invention. In principle, this second embodiment represents a similar concept as has been given and described in the first embodiment of this invention by reference to FIG. 1, with the main difference of having at least one smart sensor (4) and at least one medical data interface device (5) replacing the at least one medical device (1) given in FIG. 1. In this sense the description relating to sensors, measured signals, diagnosis results, syndrome analysis, memory components, pre-processing tasks, microprocessor tasks, types of generated results, analysis and/or reports, displays, input elements, data-interfaces to computers, software applications used for diagnosis purposes, types of MSD connectivity and other related descriptions of components, purposes and means as given in the description to the first embodiment also apply to the second embodiment, whereas the respective components of the medical device (1) of the first embodiment are found distributed in the smart sensors (4) and medical data interface device (5) in the second embodiment.

The smart sensors (4) each comprises at least one of the respective sensors for measuring signals taken from a patient or at a patient's location. Such measurements are recorded and stored, at least partly, by each smart sensor (4), possibly in a pre-processed format, in a memory which is comprised by each smart sensor (4). For possible pre-processing the smart sensors can also comprise a microprocessor. Such pre-processing can involve signal filtering and noise cancellation, event counting etc. One possible purpose of the pre-processing would be to ensure that data are recorded and stored in such a way that all the relevant information for the intended use of the medical system becomes available and fits in the smart sensor's memory. This could include reducing data size by data compression, band-width limitations and noise cancellations or adapting sampling rates to allow higher signal resolution during events of interest. This could also include high-resolution time-stamping of events for later data synchronization and overall preparation of data for transfer to the medical data interface device (5). Each smart sensor (4) comprises a data interface which offers connectivity with a medical data interface device (5), see FIG. 2b. After acquiring measurements by the smart sensors (4) for diagnosis purposes, see FIG. 2a, the smart sensors (4) are connected to the medical data interface device (5) according to FIG. 2b, and measurement data, possibly in respective pre-processed form generated in the smart sensor (4), are transferred from the smart sensor (4) to a memory component comprised by the medical data interface device (5), for later analysis and visualization. For this purpose, the medical data interface device (5) comprises at least one data interface which allows connectivity with the respective smart sensors (4) according to FIG. 2b. By this connectivity the medical data interface device (5) can also be used to access the memory of the respective smart sensors (4), in order to configure settings of the respective smart sensors (4) prior to and according to the intended diagnosis measurement and analysis procedure. This could include synchronizing smart-sensors real-time clocks, synchronizing start times and durations of recordings, selecting the appropriate settings of the recording such as sampling-rate, pre-processing settings, event monitoring, data formats etc.

The medical data interface device (5) comprises at least one microprocessor for supporting various tasks such as data processing, data transfer and possibly also visualization. The corresponding microprocessor can be selected powerful enough to perform complex analysis of respective collected data and to generate results, such as analysis and/or reports which are stored in electrical form in the respective memory component of the medical data interface device (5). As shown in FIG. 2b, the medical data interface device (5) can also comprises a display (6) for visualization; this can be used by the operator to review data, analysis and results, and possibly also reports which have been generated and/or stored in the medical data interface device's (5) memory. The medical data interface device (5) comprises a further data interface for allowing connectivity with a computer as illustrated in FIG. 2c, which enables the same features as those describe in above as being applicable when the medical device (1) is connected to a computer (3), see FIG. 1c. By this way, the medical data interface device (5) becomes accessible by the computer (3) as an MSD, when connected to the respective computer according to FIG. 2c. It follows that for the case of connecting the medical data interface device (5) to the computer (3), according to FIG. 2c, the details on the possible use and implementation of related software applications, and corresponding possibilities of performing analysis, generating results, visualization amongst other functionalities are analogous to those described in above for the first embodiment of this invention for the use of connecting the computer (3) to the medical device (1) as illustrated in FIG. 1c.

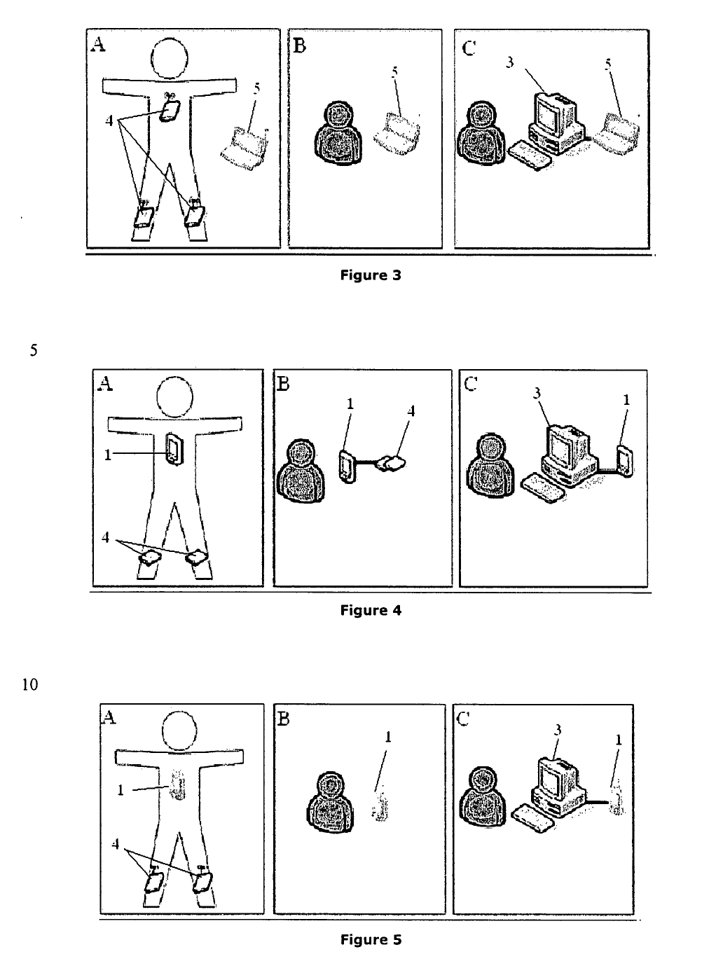

FIG. 3 shows the third possible embodiment of the present invention which is analogous to the second embodiment described by FIG. 2, with the only difference that the connection being used to connect the smart sensors (4) to the respective data interface device (5) allows a continuous connectivity between the smart sensors (4) and the medical data interface device (5), also during the time-phase when the smart sensors (4) are collecting measurements from the patient or at the patient's location. In such a constellation the analysis, visualization and other functionalities which can only take place in the second embodiment when all measurements have been collected by the smart sensors (4), see FIG. 2b and 2c, can now take place on-the-fly, during the process of the measurement. FIG. 3 illustrates for this purpose the use of a wireless link, such as for example a Bluetooth connection, WLAN connection, infrared connection or radio-links, existing between the smart sensors (4) and the medical data interface device (5), while a continuous connectivity can obviously also be provided by wired solutions. In the same manner, the data link between smart sensors (4) and the medical data interface device (5) provided for in the second embodiment as illustrated in FIG. 2, can either be implemented by wired or wireless data communication technologies. A useful purpose of implementing continuous streaming of data is to simplify the operation of the medical system by eliminating the need of post-measurement loading of sampled data to the medical data interface device (5). Yet another possible purpose is to let events detected in one smart sensor (4) and/or medical data interface device (5) influence the operation of other smart sensors (4) and/or medical data interface devices (5), such as by changing operation mode, sampling rate etc. Furthermore, a continuous connectivity allows a more efficient and accurate implementation of data synchronization, as it eliminates the inaccuracy caused by different drifting of internal real time clocks or other time keeping mechanisms possibly comprised by the smart sensors and/or medical data interface devices (5).

Synchronization of data, such as sampled measurement data, is one of the most important features of medical diagnoses and can be critical for accurate diagnoses of specific diseases. Medical symptoms are often identified by analyzing the time difference between events in the human body which must be viewed by extracting chronological relations. Examples of such approaches can be found in the detection of hypopnea in sleep monitoring, where a reduction in breathing airflow follows the oxygen desaturation of the blood. Another example is the calculation of pulse transit time (PTT) and arousal connected with body events.

Connectivity can be provided by wireless or wired connections, whereby the wireless systems are obviously more convenient in many ways. Cables used for wired connections can represent a source of unreliability and clearly cause more inconveniences to patients due to their nature. Completely wireless solutions suffer from the fact that they must have an internal powersource which may result in impractical size and weight of devices. Other aspects, such as reliability issues due to noise and interferences inherent in wireless communication systems, can play a significant role when selecting appropriate means of communications to be implemented in medical systems. It follows that all wired or wireless connections as given in described embodiments of this inventions, see FIGS. 1-5, have been selected for demonstration purposes only, and should not be considered as representing a limiting factor. In this sense, in each of the FIGS. 1-5, a wired link could be replaced by a wireless link or vice verse, depending on the underlying case of the medical system application, viewed in comparison with the various aspects such as described in above.

FIGS. 4 and 5 show related embodiments of this invention, in which an extended medical device (6) seen in FIG. 4a represents the medical device (1) according to the first embodiments illustrated in FIG. 1, but with the extension of a further data interface being comprised by the extended medical device (6) for allowing connectivity with the smart sensors (4), as shown in FIG. 4a and FIG. 4b. The respective smart sensors (4) have the same characteristics and features as those described in the foregoing descriptions of possible embodiments of the invention using smart sensors (4). By this approach the conceptual advantages, as described in above as provided by the use of medical data interface devices (5) and medical devices (1) have been incorporated in a single medical system illustrated in FIGS. 4 or 5. In the same manner as given in the foregoing descriptions of embodiments of the invention, the constellation of medical systems which use extended medical devices (6) can be based on continuous connectivity or not, as illustrated in FIGS. 5 and 4 respectively.

1. A medical system for collecting, storing and analyzing measurement data obtained from a patient or at a patient's location for diagnosis purposes, comprising at least one medical device which comprises a memory component and a microprocessor, and said system comprises at least one sensor connected to said device, said sensor placed on a patient or at a patient's location for acquiring measurement data, wherein said microprocessor pre-processes and/or stores said acquired measurement data in said memory component in raw and/or pre-processed format, the said medical device furthermore comprises at least one data communication port for exchanging data, such as said measurement data, between the said medical device and a computer, whereby upon connectivity the said medical device appears as a mass storage device on the computer.

2. A medical system according to claim 1, wherein the said data exchanged between the said medical device and said computer comprises analysis and/or diagnosis results, such as possibly reports, which are generated by said microprocessor of the said medical device for diagnosis purposes and are stored in said memory of the medical device.

3. A medical system according to claim 1, wherein the said computer executes software for performing analysis of data provided by said medical device, wherein the said software is stored in the said memory of the medical device.

4. A medical system according to claim 1, wherein the said computer executes software for performing analysis of data provided by said medical device, wherein the said software is stored in the computer.

5. A medical system according to claim 3, wherein the said software is automatically executed by said computer when the respective medical device is connected to the said computer by way of respective communication ports.

6. A medical system according to claim 3, wherein the said analysis of data provided by said computer is based on performing a combination and/or synchronisation of data and/or results stored in memory of at least one medical device.

7. A medical system according to claim 1, wherein the said medical device comprises a display component for displaying data stored in said memory of the medical device to the medical device's operator.

8. A medical system according to claim 7, wherein the said medical device comprises input elements used by said operator for interactive and/or selective viewing of respective data.

9. A medical system according to claim 1, wherein said data communication port is based on wireless communication technologies.

10. A Medical System according to claim 1, wherein the said diagnosis purpose refers to measurement results regarding blood-pressure.

11. A Medical System according to claim 1, wherein the said diagnosis purpose refers to measurement results regarding quality of sleep and/or sleep disorders

12. A Medical System according to claim 11, wherein the said diagnosis is based on analysis of at least one disease, disorder or condition selected from
Apnea/Hypapnea
Respiratory Flow Limitation
RLM
Snoring
Activity
Heart Rate Variations
High/low blood pressure.

13. A Medical System according to claim 1, wherein at least one of said sensors located on a patient or at patient's location comprise at least one sensor selected from
Respiratory effort belts sensors
Body Position signal/activity sensors
Light detection sensor
Snoring sound sensor
Snoring vibration sensor
Pressure cannula sensor
Mask pressure sensor
EMG sensor
LM Vibration sensor
ECG sensor
Pulse Oximeter
EEG/EOG sensor
Flow generator digital or analog output sensor
Bruxism sound or vibration sensor
Blood Pressure sensor.

14. A medical system for collecting, storing and analyzing measurement data obtained from a patient or at a patient's location for diagnosis purposes, comprising at least one smart sensor which comprises a memory component, a microprocessor and comprises at least one sensor placed on a patient or at a patient's location for acquiring measurement data, whereby said microprocessor pre-processes and/or stores said acquired measurement data in said memory component in raw and/or pre-processed format, the said smart sensor furthermore comprises at least one data communication port for exchanging data between the said smart sensor and a medical data interface device comprised in said medical system, whereby the said medical data interface device comprises a memory component, a microprocessor and at least one data communication port for exchanging data between the said smart sensor and the said medical data interface device, and at least one further data communication port for exchanging data between the said medical data interface device and a computer, whereby upon connectivity with the respective computer the said medical data interface device appears as a mass storage device on the computer.

15. A medical system according to claim 14, wherein the said data exchanged between the said medical data interface device and said computer comprises analysis and/or diagnosis results, such as possibly reports, which are generated by said microprocessor of the said medical data interface device for diagnosis purposes and are stored in said memory of the medical data interface device.

16. A medical system according to claim 14, wherein the said computer executes software for performing analysis of data provided by said medical data interface device, wherein the said software is stored in the said memory of the medical data interface device.

17. A medical system according to claim 14, wherein the said computer executes software for performing analysis of data provided by said medical data interface device, wherein the said software is stored in the computer.

18. A medical system according to claim 16, wherein the said software is automatically executed by said computer when the respective medical data interface device is connected to the said computer by way of respective communication ports.

19. A medical system according to claim 16, wherein the said analysis of data provided by said medical data interface device is based on performing a combination and/or synchronisation of data and/or results stored in memory of the medical data interface device and originating from measurements collected by at least one of said smart sensors.

20. A medical system according to claim 14, wherein the said medical data interface device and/or the said smart sensor comprises a display component for displaying data stored in said memory of the respective medical data interface device and/or smart sensor to the medical device's operator.

21. A medical system according to claim 20, wherein the said medical data interface device and/or smart sensors comprise input elements used by said operator for interactive and/or selective viewing of respective data.

22. A medical system according to claim 14, wherein at least one of said data communication ports is based on wireless communication technologies.

23. A Medical system according to claim 14, wherein the said diagnosis purpose refers to measurement results regarding blood-pressure.

24. A Medical system according to claim 14, wherein the said diagnosis purpose refers to measurement results regarding quality of sleep and/or sleep disorders.

25. A Medical System according to claim 24, wherein the said diagnosis is based on analysis of at least one disease, disorder or condition selected from
Apnea/Hypapnea
Respiratory Flow Limitation
RLM
Snoring
Activity
Heart Rate Variations
High/low blood pressure.

26. A Medical System according to claim 14, wherein at least one of said sensors located on a patient or at patient's location comprise at least one sensor selected from
Respiratory effort belts sensors
Body Position signal/activity sensors
Light detection sensor
Snoring sound sensor
Snoring vibration sensor
Pressure cannula sensor
Mask pressure sensor
EMG sensor
LM Vibration sensor
ECG sensor
Pulse Oximeter
EEG/EOG sensor
Flow generator digital or analog output sensor
Bruxism sound or vibration sensor
Blood Pressure sensor.

27. A medical system according to claim 1, wherein the said medical device comprises a further data communication port for connecting with at least one smart sensor which is comprised by the respective medical system and comprises a memory component, a microprocessor and comprises at least one sensor placed on a patient or at a patient's location for acquiring measurement data, whereby said smart sensor's microprocessor pre-processes and/or stores respective acquired measurement data in said memory component in raw and/or pre-processed format, the said smart sensor furthermore comprises at least one data communication port for exchanging data between the said smart sensor and said medical device.
